SEQUENCE LISTING (64 sequences)

SEQ ID NO: 1 - protein, 563 aa, Ajuga reptans
  source 1..563: note = an amino acid sequence coded by DWF1 gene from Ajuga reptans
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTVPMNLSLAVVAELDDLTVGGLINGYGIEGSSHLYGLFSDTVVSYEIVLADGRLVRATKDNQYSDLFYAIPWSQGTLGLLVAAEVKLIPVKEYMKVTYKPIVGNLKELAQGYIDSFAPRDGDQDNPEKVPDFVETMIYSPTEGVCMTGRYASKEEAKKKGNKINSVGWWFKPWFYQHAQTALKKGEFVEYIPTRDYYHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 2 - protein, 563 aa, synthetic construct
  source 1..563: note = A sterol delta 24(28) reductase, ArV143G, having its valine at position 143 mutated to glycine relative to the wild type
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTGPMNLSLAVVAELDDLTVGGLINGYGIEGSSHLYGLFSDTVVSYEIVLADGRLVRATKDNQYSDLFYAIPWSQGTLGLLVAAEVKLIPVKEYMKVTYKPIVGNLKELAQGYIDSFAPRDGDQDNPEKVPDFVETMIYSPTEGVCMTGRYASKEEAKKKGNKINSVGWWFKPWFYQHAQTALKKGEFVEYIPTRDYYHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 3 - protein, 563 aa, synthetic construct
  source 1..563: note = A sterol delta 24(28) reductase, ArM235T, having its methionine at position 235 mutated to threonine relative to the wild type
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTVPMNLSLAVVAELDDLTVGGLINGYGIEGSSHLYGLFSDTVVSYEIVLADGRLVRATKDNQYSDLFYAIPWSQGTLGLLVAAEVKLIPVKEYTKVTYKPIVGNLKELAQGYIDSFAPRDGDQDNPEKVPDFVETMIYSPTEGVCMTGRYASKEEAKKKGNKINSVGWWFKPWFYQHAQTALKKGEFVEYIPTRDYYHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 4 - protein, 563 aa, synthetic construct
  source 1..563: note = A sterol delta 24(28) reductase, ArS306P, having its serine at position 306 mutated to proline relative to the wild type
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTVPMNLSLAVVAELDDLTVGGLINGYGIEGSSHLYGLFSDTVVSYEIVLADGRLVRATKDNQYSDLFYAIPWSQGTLGLLVAAEVKLIPVKEYMKVTYKPIVGNLKELAQGYIDSFAPRDGDQDNPEKVPDFVETMIYSPTEGVCMTGRYASKEEAKKKGNKINPVGWWFKPWFYQHAQTALKKGEFVEYIPTRDYYHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 5 - protein, 563 aa, synthetic construct
  source 1..563: note = A sterol delta 24(28) reductase, ArY338H, having its tyrosine at position 338 mutated to histidine relative to the wild type
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTVPMNLSLAVVAELDDLTVGGLINGYGIEGSSHLYGLFSDTVVSYEIVLADGRLVRATKDNQYSDLFYAIPWSQGTLGLLVAAEVKLIPVKEYMKVTYKPIVGNLKELAQGYIDSFAPRDGDQDNPEKVPDFVETMIYSPTEGVCMTGRYASKEEAKKKGNKINSVGWWFKPWFYQHAQTALKKGEFVEYIPTRDYHHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 6 - protein, 563 aa, synthetic construct
  source 1..563: note = A sterol delta 24(28) reductase, Ar207, having its valine at position 143 mutated to glycine, serine at position 306 mutated to proline, and tyrosine at position 338 mutated to histidine relative to the wild type
MSDLEVPLRPKRKKIWVDYFVQFRWIIVIFLVLPISFTLYFLTYLGDVRSESKSFKQRQREHDENVEKVVKRLKERNPKKDGLVCTARKPWISVGMRNVDYKRARHFEVDLSAFRNVLDIDKERMIARVEPLVNMGQISRVTGPMNLSLAVVAELDDLTVGGLINGYGIEGSSHLYGLFSDTVVSYEIVLADGRLVRATKDNQYSDLFYAIPWSQGTLGLLVAAEVKLIPVKEYMKVTYKPIVGNLKELAQGYIDSFAPRDGDQDNPEKVPDFVETMIYSPTEGVCMTGRYASKEEAKKKGNKINPVGWWFKPWFYQHAQTALKKGEFVEYIPTRDYHHRHTRSLYWEGELILPFADQWWFRFLLGWLMPPKVSLLKATQGEAIRNYYHEMHVIQDMLVPLYKVGDALEFVHREMELYPVWLCPHRLFKLPVKPMISPEPGFELQRRQGDTHYAQMYTDILLCYAPGPVLRGEQFDGAEAVHRMENWLIENHGYEPQYTVSELSEKNFWRMFDGGLYEQCRRKYGAVGTFMSVYYKSKKGRKTEKEVQEAEQAILESPDAEVA

SEQ ID NO: 7 - DNA, 7781 bp, synthetic construct
  source 1..7781: mol_type = other DNA; note = A nucleotide sequence for an expression cassette of ARE2, YEH1, and YEH2 genes
atgagaaatatcgagggactcgattcagaggagcaggacaaactataaccgactgtttgttggaggatgccgtacataacgaacactgctgaagctaccatgtctacagtttagaggaatgggtacaactcacaggcgagggatggtgttcactcgtgctagcaaacgcggtgggagcaaaaagtagaatattatcttttattcgtgaaacttcgaacactgtcatctaaagatgctatatactaatataggcatacttgataatgaaaactataaatcgtaaagacataagagatccgctcaagccttctcagcaaccatttccagctccgtttccacctctgtgtccgaacaatcactttcaaaggcgctggatggcttcttggagtaatagttgttcaaattctgtaaaattggtttaccaattctctctatgacatcatgtgcccataggacatcaatatgggcatactcatcaatgtaccagatcttgtagttgacattcgactccacattgacgaaatgattgattagtctttctccgtccaccaatctatcttgctttggaacaaacaagtagatatttggggcctttgatgcgtctgaaaaccacttaacattgtcggggaacatcttatgggaaccaaatttaaaacttaccttgttggggtcgggtgacagccaccattgcatcaacttcactgaaacatggactggcgaaaacaggaaatggcgatctcttaatgcggtatcccaaagggtatcgttccaatcaaacaggtaattgaagatggtgtaacaaacaaaggagaacaagctctcaccaacgcacaagtttcttacaatcatcattatctcaaaaaagctcgtttcaccaaagaaccagggattttcgatttcctttgtcataagcttaacaaacaatttctcgttaagtaagggaccaggatacactgcgggggccaaggcaatatagtttgcgatcttagaagtgaaaaaagattctttagatcccgaaccgggagggaaaaacttatcttcgttgaccaagcccataaacccctgtgtagtgccctgagaatgcgagatcaaagtaagcttttcaaactgcgtcttagctaacacggtatcaatcaaaagggtcagatcgtacttaaccatctcgcgaaggtcccagtcccacctggaagctagtgtcggtactttcgcttcgttccattccggcctaaacccgcatctgttattccctaaccatatgtcgtaaccggattgatacagaaaatatgccagagattttctaccattggatgcgaacgaaccactgctttgcaaaaggccatgtagcatcaaaatgggtggcctcttcttcttgtcagaatctgtcgttctatattttggtatcaagtgccacaaatctataacaaacccatcttcggtctccaatctaaattcctctatttgaatattgtactggttaaaataataattaagatccgcaactaatgtcaagtcttccgataagattagattctgtaatggattgtcgcactgtacaggaattttttcaaaaaccggttcatgattggcaatttcattgctactattattgaactttccttgcgttacgccttcggtaactttttccttctcgataaacctaaaaataagctttgtcaaaaatttgtgaaaaaggtttttatgcagtgacgaggaccccacttcgtggttctttttacggacaaccgttcttgtatgatcgtagacgataccgtttttcttgtcatgttccagatcgaggtatggcgtcgcgtatgcattggttctagtgagaacatggtcatggtgtacgtttcttttactttcgtcatcaacttcgatttgggtcgatgaactcctagtgtctctgtgctcatttataaaatggtgtgccagcgccagaacaagcactactgccataaaacagcagactatgaacgttgctagtaaattcctagctcttttcaacaccgcagaaacacccatctagaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatgcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggacaagaagaaggatctactggagaacgaacaatttctccgcatccaaaagctcaacgctgccgatgcgggcaaaagacaatctataacagtggacgacgagggcgaactatatgggttagacacctccggcaactcaccagccaatgaacacacagctaccacaattacacagaatcacagcgtggtggcctcaaacggagacgtcgcattcatcccaggaactgctaccgaaggcaatacagagattgtaactgaagaagtgattgagaccgatgataacatgttcaagacccatgtgaagactttaagctccaaagagaaggcacggtataggcaagggtcctctaactttatatcgtatttcgatgatatgtcatttgaacacaggcccagtatattagatgggtcagttaacgagcccttcaagaccaaattcgtgggacctactttagaaaaggagatcagaagaagggagaaagagctaatggccatgcgcaaaaatttacaccaccgcaagtcctccccagatgctgtcgactcagtagggaaaaatgatggcgccgccccaactactgttccaactgccgccacctcagaaacggtggtcaccgttgaaaccaccataatttcatccaatttctccgggttgtacgtggcgttttggatggctattgcatttggtgctgtcaaggctttaatagactattattaccagcataatggtagcttcaaggattcggagatcttgaaatttatgactacgaatttgttcactgtggcatccgtagatcttttgatgtatttgagcacttattttgtcgttggaatacaatacttatgcaagtggggggtcttgaaatggggcactaccggctggatcttcacctcaatttacgagtttttgtttgttatcttctacatgtatttaacagaaaacatcctaaaactacactggctgtccaagatcttcctttttttgcattctttagttttattgatgaaaatgcattctttcgccttctacaatggctatctatggggtataaaggaagaactacaattttccaaaagcgctcttgccaaatacaaggattctataaatgatccaaaagttattggtgctcttgagaaaagctgtgagttttgtagttttgaattgagctctcagtctttaagcgaccaaactcaaaaattccccaacaatatcagtgcaaaaagttttttttggttcaccatgtttccaaccctaatttaccaaattgaatatccaagaactaaggaaatcagatggagctacgtattagaaaagatctgcgccatcttcggtaccattttcttaatgatgatagatgctcaaatcttgatgtatcctgtagcaatgagagcattggctgtgcgcaattctgaatggactggtatattggatagattattgaaatgggttggattgctcgttgatatcgtcccagggtttatcgtgatgtacatcttggacttctatttgatttgggatgccattttgaactgtgtggctgaattgacaagatttggcgacagatatttctacggtgactggtggaattgtgttagttgggcagacttcagtagaatttggaacatcccagtgcataagtttttgttaagacatgtttaccatagttcaatgagttcattcaaattgaacaagagtcaagcaactttgatgacctttttcttaagttccgtcgttcatgaattagcaatgtacgttatcttcaagaaattgaggttttacttgttcttcttccaaatgctgcaaatgccattagtagctttaacaaatactaaattcatgaggaacagaaccataatcggaaatgttattttctggctcggtatctgcatgggaccaagtgtcatgtgtacgttgtacttgacattctaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatggtaaataaggtggttgatgaggttcagcggttggtgagcgcaataattctcacttcgtttatgacggggttattcattttgtcactatggaagaattacgtgacagttcatttccaacataaaaatgatccaagagatacaagaagttcgagaaccaagatccagccaaatgacaagaagaagaaacgaccagcacgtcactctagacctttgtcaatatcttccacaacgcccttagatttacagcgggatcaggaaaataatattgaatatgaccgtactgtgactagcaagctgtccatgactagtaatgcctctttatcagagaacggtgatggaaatgctaatattaaaatggaaacgaatgtgaaccaagccccatatgctgcagagaatcccttccaaaacattgcgcttgcggaggataccaagctggtacctgacttaaagtactattataaagagtatggtattgatattgaagaatttgaggtggagacagacgacggttttatcattgatttgtggcacttcaagtccaggttaaacgatggcgtagaggaagtgaaacgtgaacccattttgctattacacggtcttttgcaaagttgcggggccttcgcatcttcaggtagaaaatctctggcatacttcctttatgaatctggatttgatgtttggttggggaataacagatgtggcttaaatgccaagtggaacatgaaaaaattaggtaatgatcattccaaaaaatgggattgggatatgcaccagatggttcaatacgatttaaaagcacttataaattatgtacttgatagtaccggatatgctaaactatcgctcgtggcgcattcacaaggtacaacacaagggtttatggggcttgtcaacggtgagaaactatacgcaagtgattttaaattagtcgataagctggaaaactttgttgctttggcacctgcagtgtaccctggacctttattggatgaaaaagcatttgtgaggctaatggcaaaaggtattgattctccgtggtactttggtagaagatcgtttatacctcttatgatgacaatgagaaaactaatggttgggaccaagattttttcgttcctctcatacatcatgtttaactacctatttgattggaacgatgtcttatgggatagagttttaagggaccgaaatttcttattttcaccagtccatatttccgtcaaactaatgcaatggtggttgtcacctcttcctaataagctaagttttaagaagggtgcagagaaaatttttccagataaaaagacatggtttcctattgccaagaatgatgatgacagtggcaataatttggataacaacaaactacacttaaatccgaaaagacaaaatagtgaagaatttccacacattataatgtttattccaaagcaagatagattagtagatggtgaacgattaattaatcacttcattaatcatgaagcaaatgcagtgtacaaaatctggtatattgatgagtattcccatctggacgttttatgggcgcatgatgttatagatagaattggtaagccaatgatagaaaatttgaggtttcctaatgcaaggtgaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaa

SEQ ID NO: 8 - DNA, 2660 bp, synthetic construct
  source 1..2660: mol_type = other DNA; note = A nucleotide sequence for an expression cassette of PGAL10-PGAL1-Ar207
ttatattgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatgtccgatttggaagttccattgaggccaaagcgtaagaagatttgggttgactacttcgtccaattcagatggattattgttatcttcttggttttgccaatttctttcacattgtactttttaacctacttgggcgacgttagatctgaatccaagtctttcaaacaaagacaaagagaacacgatgaaaacgttgaaaaagttgttaagagattgaaagaaagaaatcctaagaaggatggtttggtttgtaccgctcgtaaaccatggatttctgtcggtatgagaaacgttgattataaaagagccagacattttgaagtcgatctatctgccttcagaaatgttttggacatcgataaggaaagaatgattgctagagttgaaccattagttaacatgggtcaaatctccagagttaccgttccaatgaatctgtccttggctgttgttgctgaattggatgatcttaccgtcggtggtttaattaacggttacggtatcgaaggttcttctcacttgtatggtttattttctgatactgttgtctcctacgaaatagttttagctgacggtagattagttcgtgctactaaagataaccaatactctgacttgttctatgcaattccatggtctcaaggtactttaggtttgttggttgctgctgaagttaaattgattccagttaaggaatacatgaaggtcacatataaacctattgttggtaatctaaaggagttagctcaaggttatattgattctttcgctccaagagatggtgaccaagataatccagaaaaggttcctgactttgttgaaactatgatctattcaccaactgaaggtgtttgtatgactggtaggtacgcttcaaaggaagaagctaagaagaagggtaacaagatcaactctgttggttggtggttcaaaccatggttctaccaacatgctcaaacagctttaaaaaagggtgaattcgttgaatatattccaactagagattattatcatagacatacccgttctttgtactgggaaggtgaattgattttgccttttgctgatcaatggtggttccgtttcttgttaggttggttaatgcctcctaaggtttctttgttgaaggccactcaaggtgaagcaattagaaactactaccatgaaatgcatgttattcaagatatgctagttccattatataaagttggagatgctttagaatttgttcatcgtgaaatggaattgtacccagtttggttatgtccacacagattgtttaagttacctgttaagccaatgattagtccagaaccaggtttcgaattgcaacgtagacaaggtgacactcattacgctcaaatgtacactgatattttgttgtgttacgctccaggtccagtgttgagaggtgaacaatttgatggtgctgaagctgttcatagaatggaaaactggttaattgaaaaccacggttatgaaccacaatacactgtttctgaattgtctgaaaagaacttctggagaatgtttgatggtggtttgtatgaacaatgtagacgtaagtacggtgctgtaggtacttttatgtctgtttattataagtctaagaagggtagaaagactgaaaaagaagttcaagaagctgaacaagctattttggaatctccagatgctgaagttgcttaagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgagtccctcgatatttctcat

SEQ ID NO: 9 - DNA, 2942 bp, synthetic construct
  source 1..2942: mol_type = other DNA; note = A nucleotide sequence for an ERG5 expression cassette
catagcttcaaaatgtttctactccgaccaatgttaatgaaaacttgaactgctgaagtgtgatattcaatgtatgagataatccattaaatcatctacagtttaaagactgtaggcttacatgaccgtttatcactgcgatattggaggaagcgcttcggatcaatggaccttttacttagcagagcgaaggctgaaaatcatgaaccccaacagcatgtgtgacctctcttctcacgcctggcaagaaaccaaactacggcatacattcagcgcaccaaaaaggaactccgtactatttacattaaaaacaagaaaatgtgttgccatttccgtaaaatgaatggcccatgttcatacgcgtatgcaaccgtcttatttacactccgactaatgtccttctaattactgtggaagacggccgtataagcgtccttctgtggtttagatatgctataccggcggaactttgttacacacggctcgcgcgaatccttaggggaaaacattgcgctgactttccccagagttgttgccacaacataagccgctttggagtgttgaacaaatccgtccttgggtcattcaatcaatggcttggcggtatctcaaaagagcgcaaactaatagcgcgcacattcgacgcatttatccggtggtcatcgactaggggcgaagaggtcacgacctattttttcttgcagaaaaaaagtgtgaccttttccgtagctagacgtctatcagggcgtcagcaatgggaggcacagcggaaaaacaataacaatggtaagcgcaattaccttttgagcgttacattcgtatgaaattggtgacgttaatctaaagatagtcatgctctcaaaagggcccattattctcgacgttgagcgtatataagactattaaaacttggttctttagatatggtgttcgttcctcattattaagtttcagggaacaatatcaacacatatcataacaggttctcaaaactttttgttttaataatactagtaacaagaaaaatgagttctgtcgcagaaaatataatacaacatgccactcataattctacgctacaccaattggctaaagaccagccctctgtaggcgtcactactgccttcagtatcctggatacacttaagtctatgtcatatttgaaaatatttgctactttaatctgtattcttttggtttgggaccaagttgcatatcaaatcaagaaaggttccatcgcaggtccaaagtttaagttctggcccatcatcggtccatttttggaatccttagatccaaagtttgaagaatataaggctaagtgggcatccggtccactttcatgtgtttctattttccataaatttgttgttatcgcatctactagagacttggcaagaaagatcttgcaatcttccaaattcgtcaaaccttgcgttgtcgatgttgctgtgaagatcttaagaccttgcaattgggtttttttggacggtaaagctcatactgattacagaaaatcattaaacggtcttttcactaaacaagctttggctcaatacttaccttcattggaacaaatcatggataagtacatggataagtttgttcgtttatctaaggagaataactacgagccccaggtctttttccatgaaatgagagaaattctttgcgccttatcattgaactctttctgtggtaactatattaccgaagatcaagtcagaaagattgctgatgattactatttggttacagcagcattggaattagtcaacttcccaattattatcccttacactaaaacatggtatggtaagaaaactgcagacatggccatgaagattttcgaaaactgtgctcaaatggctaaggatcatattgctgcaggtggtaagccagtttgtgttatggatgcttggtgtaagttgatgcacgatgcaaagaatagtaacgatgatgattctagaatctaccacagagagtttactaacaaggaaatctccgaagctgttttcactttcttatttgcttctcaagatgcctcttcttctttagcttgttggttgttccaaattgttgctgaccgtccagatgtcttagctaagatcagagaagaacaattggctgttcgtaacaatgacatgtctaccgaattgaacttggatttgattgagaaaatgaagtacaccaatatggtcataaaagaaactttgcgttacagacctcctgtcttgatggttccatatgttgttaagaagaatttcccagtttcccctaactataccgcaccaaagggcgctatgttaattccaaccttatacccagctttacatgatcctgaagtttacgaaaatcctgatgagttcatccctgaaagatgggtagaaggctctaaggctagtgaagcaaagaagaattggttggtttttggttgtggtccacacgtttgcttaggtcaaacatatgtcatgattaccttcgccgctttgttgggtaaatttgcactatatactgatttccatcatacagtgactccattaagtgaaaaaatcaaggttttcgctacaattttcccaaaagatgatttgttactgactttcaaaaagagagacccaattactggagaagtcttcgaataagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgagtccctcgatatttctcat

SEQ ID NO: 10 - DNA, 26 bp, synthetic construct
  source 1..26: mol_type = other DNA; note = a sequence for a forward primer, erg4delta-U-F
caaaactcactcctggtaatgtctat

SEQ ID NO: 11 - DNA, 36 bp, synthetic construct
  source 1..36: mol_type = other DNA; note = a sequence for a reverse primer, erg4delta-U-R
tcatcccaatccgtatctgcctatctacataactga

SEQ ID NO: 12 - DNA, 37 bp, synthetic construct
  source 1..37: mol_type = other DNA; note = A sequence for a forward primer, erg4delta-D-F
gcagatacggattgggatgagtattgtaaacattgcc

SEQ ID NO: 13 - DNA, 22 bp, synthetic construct
  source 1..22: mol_type = other DNA; note = A sequence for a reverse primer, erg4delta-D-R
tcatgggagaaggtggtagaat

SEQ ID NO: 14 - DNA, 75 bp, synthetic construct
  source 1..75: mol_type = other DNA; note = A nucleotide sequence for primer Dor.-16Ty1-PTEF1, nucleotides from positions 1 to 50 represent an homology arm sequence
cccgaccttccattttttttttcttggaatcagtacatagcaggtatgagcatagcttcaaaatgtttctactcc

SEQ ID NO: 15 - DNA, 72 bp, synthetic construct
  source 1..72: mol_type = other DNA; note = A nucleotide sequence for primer Dor.-16Ty1-PADH2, nucleotides from positions 1 to 50 represent an homology arm sequence
tacaccactttctcaaataaattcgttaaataacggtgtgttgaaatgttatgagaaatatcgagggactcg

SEQ ID NO: 16 - DNA, 41 bp, synthetic construct
  source 1..41: mol_type = other DNA; note = A nucleotide sequence for primer VArDWF1-F
atctaatctaagttttctagatgtccgatttggaagttcca

SEQ ID NO: 17 - DNA, 41 bp, synthetic construct
  source 1..41: mol_type = other DNA; note = A nucleotide sequence for primer VArDWF1-R
taaagacataagagatccgcttaagcaacttcagcatctgg

SEQ ID NO: 18 - DNA, 56 bp, synthetic construct
  source 1..56: mol_type = other DNA; note = A nucleotide sequence for primer Ep-ArDWF1-F
aaagaaagcatagcaatctaatctaagttttctagatgtccgatttggaagttcca

SEQ ID NO: 19 - DNA, 56 bp, synthetic construct
  source 1..56: mol_type = other DNA; note = A nucleotide sequence for primer Ep-ArDWF1-R
gaaaactataaatcgtaaagacataagagatccgcttaagcaacttcagcatctgg

SEQ ID NO: 20 - DNA, 26 bp, synthetic construct
  source 1..26: mol_type = other DNA; note = A nucleotide sequence for primer Ar-V143G-F
tctccagagttaccggtccaatgaat

SEQ ID NO: 21 - DNA, 26 bp, synthetic construct
  source 1..26: mol_type = other DNA; note = A nucleotide sequence for primer Ar-V143G-R
ccggtaactctggagatttgacccat

SEQ ID NO: 22 - DNA, 26 bp, synthetic construct
  source 1..26: mol_type = other DNA; note = A nucleotide sequence for primer Ar-M235T-F
cagttaaggaatacacgaaggtcaca

SEQ ID NO: 23 - DNA, 26 bp, synthetic construct
  source 1..26: mol_type = other DNA; note = A nucleotide sequence for primer Ar-M235T-R
gtgtattccttaactggaatcaattt

SEQ ID NO: 24 - DNA, 26 bp, synthetic construct
  source 1..26: mol_type = other DNA; note = A nucleotide sequence for primer Ar-S306P-F
ggtaacaagatcaaccctgttggttg

SEQ ID NO: 25 - DNA, 26 bp, synthetic construct
  source 1..26: mol_type = other DNA; note = A nucleotide sequence for primer Ar-S306P-R
ggttgatcttgttacccttcttctta

SEQ ID NO: 26 - DNA, 26 bp, synthetic construct
  source 1..26: mol_type = other DNA; note = A nucleotide sequence for primer Ar-Y338H-F
ccaactagagattatcatcatagaca

SEQ ID NO: 27 - DNA, 26 bp, synthetic construct
  source 1..26: mol_type = other DNA; note = A nucleotide sequence for primer Ar-Y338H-R
gataatctctagttggaatatattca

SEQ ID NO: 28 - DNA, 42 bp, synthetic construct
  source 1..42: mol_type = other DNA; note = A nucleotide sequence for primer pEB-TADH2-R
gcgaatgcatctagatatcgatgagaaatatcgagggactcg

SEQ ID NO: 29 - DNA, 36 bp, synthetic construct
  source 1..36: mol_type = other DNA; note = A nucleotide sequence for primer pEB-PTEF1-F
actcgaactgcatagcttcaaaatgtttctactcct

SEQ ID NO: 30 - DNA, 34 bp, synthetic construct
  source 1..34: mol_type = other DNA; note = A nucleotide sequence for primer pEB-PTDH3-F
tgaagctatgcagttcgagtttatcattatcaat

SEQ ID NO: 31 - DNA, 42 bp, synthetic construct
  source 1..42: mol_type = other DNA; note = A nucleotide sequence for primer pEB-PTDH3-R
cttaagtctttgctcgagtttgtttgtttatgtgtgtttatt

SEQ ID NO: 32 - DNA, 36 bp, synthetic construct
  source 1..36: mol_type = other DNA; note = A nucleotide sequence for primer pEB-TCYC1-F
ctcgagcaaagacttaagacaggccccttttccttt

SEQ ID NO: 33 - DNA, 28 bp, synthetic construct
  source 1..28: mol_type = other DNA; note = A nucleotide sequence for primer pEB-TCYC1-R
gtattgttattgttacatgcgtacacgc

SEQ ID NO: 34 - DNA, 34 bp, synthetic construct
  source 1..34: mol_type = other DNA; note = A nucleotide sequence for primer pEB-PFBA1-F
gcatgtaacaataacaatactgacagtactaaat

SEQ ID NO: 35 - DNA, 43 bp, synthetic construct
  source 1..43: mol_type = other DNA; note = A nucleotide sequence for primer pEB-PFBA1-R
cctaggcaacctgaattctttgaatatgtattacttggttatg

SEQ ID NO: 36 - DNA, 42 bp, synthetic construct
  source 1..42: mol_type = other DNA; note = A nucleotide sequence for primer pEB-TPGK1-F
gaattcaggttgcctaggattgaattgaattgaaatcgatag

SEQ ID NO: 37 - DNA, 40 bp, synthetic construct
  source 1..40: mol_type = other DNA; note = A nucleotide sequence for primer VYEH1-F
atctaatctaagttttctagatgggtgtttctgcggtgtt

SEQ ID NO: 38 - DNA, 41 bp, synthetic construct
  source 1..41: mol_type = other DNA; note = A nucleotide sequence for primer VYEH1-R
taaagacataagagatccgctcaagccttctcagcaaccat

SEQ ID NO: 39 - DNA, 45 bp, synthetic construct
  source 1..45: mol_type = other DNA; note = A nucleotide sequence for primer PTDH3-ARE2
aacacacataaacaaacaaaatggacaagaagaaggatctactgg

SEQ ID NO: 40 - DNA, 48 bp, synthetic construct
  source 1..48: mol_type = other DNA; note = A nucleotide sequence for primer ARE2-TCYC1
acaaaggaaaaggggcctgtttagaatgtcaagtacaacgtacacatg

SEQ ID NO: 41 - DNA, 45 bp, synthetic construct
  source 1..45: mol_type = other DNA; note = A nucleotide sequence for primer PPBA1-YEH2
ccaagtaatacatattcaaaatggtaaataaggtggttgatgagg

SEQ ID NO: 42 - DNA, 43 bp, synthetic construct
  source 1..43: mol_type = other DNA; note = A nucleotide sequence for primer YEH2-TPGK1
cgatttcaattcaattcaattcaccttgcattaggaaacctca

SEQ ID NO: 43 - DNA, 74 bp, synthetic construct
  source 1..74: mol_type = other DNA; note = A nucleotide sequence for primer Dor.-16Ty3-1-TADH2
agcgtaacaatgcatactgacattataagcctgaacattacgagtttaagatgagaaatatcgagggactcgat

SEQ ID NO: 44 - DNA, 74 bp, synthetic construct
  source 1..74: mol_type = other DNA; note = A nucleotide sequence for primer Dor.-16Ty3-1-TPGK1
agaagtagctttcgcaaatacaccaatgcattcgttgataatttacgcagttaacgaacgcagaattttcgagt

SEQ ID NO: 45 - DNA, 24 bp, synthetic construct
  source 1..24: mol_type = other DNA; note = A nucleotide sequence for primer R-pRS42H-207-F
atgtccgatttggaagttccattg

SEQ ID NO: 46 - DNA, 28 bp, synthetic construct
  source 1..28: mol_type = other DNA; note = A nucleotide sequence for primer R-pRS42H-207-R
ggagtagaaacattttgaagctatgcac

SEQ ID NO: 47 - DNA, 54 bp, synthetic construct
  source 1..54: mol_type = other DNA; note = A nucleotide sequence for primer PGAL10-GAL1-F
cttcaaaatgtttctactccttatattgaattttcaaaaattcttacttttttt

SEQ ID NO: 48 - DNA, 48 bp, synthetic construct
  source 1..48: mol_type = other DNA; note = A nucleotide sequence for primer PGAL10-GAL1-R
ggaacttccaaatcggacattatagttttttctccttgacgttaaagt

SEQ ID NO: 49 - DNA, 80 bp, synthetic construct
  source 1..80: mol_type = other DNA; note = A nucleotide sequence for primer Dor.-PACC1-PGAL10
tcgtactccatcttctgtggagaagactcgaataagctttcttcgctcatttatattgaattttcaaaaattcttacttt

SEQ ID NO: 50 - DNA, 69 bp, synthetic construct
  source 1..69: mol_type = other DNA; note = A nucleotide sequence for primer Dor.-PACC1-TADH2
ttatccctttcgtacagtttacctgaagataaaaaacaacaaagccaattatgagaaatatcgagggac

SEQ ID NO: 51 - DNA, 46 bp, synthetic construct
  source 1..46: mol_type = other DNA; note = A nucleotide sequence for primer PCIT2-F
cttcaaaatgtttctactccgaccaatgttaatgaaaacttgaact

SEQ ID NO: 52 - DNA, 54 bp, synthetic construct
  source 1..54: mol_type = other DNA; note = A nucleotide sequence for primer PCIT2-R
ggaacttccaaatcggacatttttcttgttactagtattattaaaacaaaaagt

SEQ ID NO: 53 - DNA, 34 bp, synthetic construct
  source 1..34: mol_type = other DNA; note = A nucleotide sequence for primer R-PCIT2-R
ttttcttgttactagtattattaaaacaaaaagt

SEQ ID NO: 54 - DNA, 23 bp, synthetic construct
  source 1..23: mol_type = other DNA; note = A nucleotide sequence for primer R-TADH2-F
gcggatctcttatgtctttacga

SEQ ID NO: 55 - DNA, 43 bp, synthetic construct
  source 1..43: mol_type = other DNA; note = A nucleotide sequence for primer ERG5-F
taatactagtaacaagaaaaatgagttctgtcgcagaaaatat

SEQ ID NO: 56 - DNA, 46 bp, synthetic construct
  source 1..46: mol_type = other DNA; note = A nucleotide sequence for primer ERG5-R
taaagacataagagatccgcttattcgaagacttctccagtaattg

SEQ ID NO: 57 - DNA, 75 bp, synthetic construct
  source 1..75: mol_type = other DNA; note = A nucleotide sequence for primer Dor.-15Ty2-F
tttcagcggttaaatacgaagcgcaacaagagttagcgaaaaataagtaccatagcttcaaaatgtttctactcc

SEQ ID NO: 58 - DNA, 72 bp, synthetic construct
  source 1..72: mol_type = other DNA; note = A nucleotide sequence for primer Dor.-15Ty2-R
gaaagccccgtatgaacatctgccaatagataacagttgtctctaatttcatgagaaatatcgagggactcg

SEQ ID NO: 59 - RNA, 20 nt, synthetic construct
  source 1..20: mol_type = other RNA; note = An nucleotide sequence for ERG4-gRNA
gtagaccagtgatattcaga

SEQ ID NO: 60 - RNA, 20 nt, synthetic construct
  source 1..20: mol_type = other RNA; note = A nucleotide sequence for 16Ty1-gRNA
gttagagctgttacaagtta

SEQ ID NO: 61 - RNA, 20 nt, synthetic construct
  source 1..20: mol_type = other RNA; note = A nucleotide sequence for 16Ty3-gRNA
gtatgtaggcgttctgtaag

SEQ ID NO: 62 - RNA, 20 nt, synthetic construct
  source 1..20: mol_type = other RNA; note = A nucleotide sequence for PACC1-gRNA
gcaaatggcgattgcaaatt

SEQ ID NO: 63 - RNA, 20 nt, synthetic construct
  source 1..20: mol_type = other RNA; note = A nucleotide sequence for 15Ty2-gRNA
taagtaatggtagcgtagaa

SEQ ID NO: 64 - DNA, 41 bp, synthetic construct
  source 1..41: mol_type = other DNA; note = A nucleotide sequence for primer pEB-TPGK1-R
ctatgaccatgattacgccattaacgaacgcagaattttcg

(19) United States
(12) Patent Application Publication
(10) Pub. No.: US 2025/0270612 A1
(43) Pub. Date: Aug. 28, 2025

(54) FUNGUS SYNTHESIZING 24-EPI-ERGOSTEROL, CONSTRUCTION METHOD THEREFOR, AND USE THEREOF

(71) Applicant: NINGBO XINBIO BIOLOGICAL SCI. & TECH. CO., LTD., Ningbo (CN)

(72) Inventors: Jianping LIN, Ningbo (CN); Yiqi JIANG, Ningbo (CN); Li ZHU, Ningbo (CN); Zhijiao SUN, Ningbo (CN); Hailong XUE, Ningbo (CN); Mianbin WU, Ningbo (CN); Lirong YANG, Ningbo (CN)

(21) Appl. No.: 19/190,583

(22) Filed: Apr. 25, 2025

Related U.S. Application Data
(63) Continuation-in-part of application No. PCT/CN2023/133562, filed on Nov. 23, 2023.

(30) Foreign Application Priority Data
Nov. 28, 2022 (CN) 202211506223.6

(51) Int. Cl. C12P 33/00; C12N 1/18; C12N 9/02; C12N 15/81; C12R 1/865

(52) U.S. Cl. CPC C12P 33/005; C12N 1/185; C12N 9/001; C12N 15/81; C12Y 103/01072; C12R 2001/865

(57) ABSTRACT

Provided is a fungus synthesizing 24-epi-ergosterol, a construction method therefor, and use thereof. A sterol Δ²⁴⁽²⁸⁾ reductase DWF1 gene or a mutant gene thereof is recombinantly expressed in a fungal body producing 24(28)-dehydroergosterol fungal body producing ergosterol, and the strain synthesizing 24-epi-ergosterol is constructed. By means of directed evolution of DWF1, DWF1 has an increased ability to reduce a unpreferable substrate 24(28)-dehydroergosterol to form 24-epi-ergosterol in the fungus, and in combination with high-density fermentation, the yield of 24-epi-ergosterol is improved; by means of the use of a synthetic biology strategy that enhances esterification and hydrolysis to enhance sterol homeostasis, the metabolic flux towards 24-epi-ergosterol in the fungus is increased; the promoters of DWF1 mutant and ACC1 genes are optimized, and ERG5 is overexpressed, so that the proportion of 24-epi-ergosterol in sterols is increased, and in combination with the high-density fermentation, the yield of 24-epi-ergosterol is significantly improved.

### CROSS REFERENCE TO RELATED APPLICATION

This application is a continuation-in-part of International Application No. PCT/CN2023/133562 entitled “FUNGUS SYNTHESIZING 24-EPI-ERGOSTEROL, CONSTRUCTION METHOD THEREFOR, AND USE THEREOF” filed with the International Bureau on Nov. 23, 2023, which claims the benefit and priority of Chinese Patent Application No. 202211506223.6 filed with the China National Intellectual Property Administration (CNIPA) on Nov. 28, 2022. Therefore, the earliest priority date of this patent application is Nov. 28, 2022, and the disclosure of both prior applications are incorporated by reference herein in their entireties as part of the present application.

### REFERENCE TO SEQUENCE LISTING

A computer readable XML file entitled “GWPCTP20241208062-Sequence Listing”, that was created on Mar. 20, 2025, with a file size of about 89,086 bytes, contains the sequence listing for this application, has been filed with this application, and is hereby incorporated by reference in its entirety.

### TECHNICAL FIELD

The present disclosure relates to a fungus synthesizing 24-epi-ergosterol, a construction method therefor and use thereof. In the present disclosure, a recombinant fungal strain producing 24-epi-ergosterol is obtained by gene edition of the fungus, relating to the technical fields of synthetic biology and enzyme engineering.

### BACKGROUND

Brassinosteroids (BRs) are a class of natural steroid lactones/ketones with high physiological activity in plants, which are recognized as the sixth class of plant hormones. BRs play a vital role in plant growth processes such as seed germination, root growth, and reproduction. Meanwhile, BRs can coordinate with other plant hormones to regulate plant oxidative free radical metabolism, ethylene synthesis, and root gravitropic responses, thereby improving plant resistance to harsh environments such as drought, high salt, and extreme temperatures. As the most biologically active compound among BRs, brassinolide (BL) (FIG. 1) shows a great potential for agricultural and industrial applications.

However, the natural abundance of BL is extremely low. Even in pollen and seeds with a high content of BL, the content remains only 1 ng/g to 100 ng/g (fresh weight) and is far from meeting the demands of research and agricultural applications. Considering the structural complexity of BRs, predecessors have proposed and attempted semi-synthetic methods based on natural sterols. In the attempt to semi-synthesize BL, crinosterol isolated from a rare marine invertebrate (Crinoidea sp.) is considered to be the best synthetic precursor (FIG. 2) (M. J. Thompson, et al, Steroids, 1981 (38), 567). However, the crinosterol is even more scarce than BL and is not sufficient for industrial synthesis of BL. In addition, predecessors have also tried to use more easily available sterols as substrates. For example, ergosterol or stigmasterol are subjected to side chain oxidation to generate steraldehydes, which are linked with chiral accessories to construct side chains with the same chirality as that of BL, especially the chirality of 24-C, thereby synthesizing BL (Fung, S. and Siddall. J. B, J. Am. Chem. Soc., 1980(102), 6580; M. Sakakibara, et al, Heterocycles, 1982(17), 301; T. C. McMorris, et al, J. Chem. Soc., Perkin Trans, 1996(1), 295; A. L. Hurski, et al, Org. Biomol. Chem., 2015(13), 1446). However, due to complicated synthesis steps, high cost, and great pollution, these processes have not achieved in industrial production. Given the inaccessibility of BL, researchers have turned to developing other BRs that are relatively less active but can be produced in large quantities. So far, two BRs have been widely used in agriculture, namely 24-epi-brassinolide (EBL) and 28-homo-brassinolide (HBL) (FIG. 1). The semi-synthetic precursors of EBL and HBL are ergosterol (T. C. Mcmorris, et al, J Org Chem, 1993(58), 2338) and stigmasterol (T. C. Mcmorris, et al, Phytochemistry, 1994(36), 585), respectively (FIG. 2).

In recent years, the development of synthetic biology has provided the possibility of efficient and low-cost production of complex natural products (such as BL) by relying on microbial cell factories. However, the biosynthetic pathway of BL has not been fully elucidated (FIG. 3). Therefore, it is the most feasible and promising technical solution under the current circumstances to transform microorganisms that grow rapidly and cost effectively on cultivation through synthetic biology, such that they can synthesize sterols with the same side chains as those of crinosterol in large quantities for industrial production of BL.

BL is different from EBL, which is one of the two BRs currently produced on a large scale, in structure only in the chirality of the side chain 24-C, which is derived from ergosterol, the semi-synthetic precursor of EBL. Accordingly, the present disclosure intends to transform the ergosterol synthesis pathway in yeast through synthetic biology to synthesize 24-epi-ergosterol, which can be conveniently used to synthesize the BL. The reaction that determines the chirality of 24-C in the ergosterol synthesis pathway in yeast is the asymmetric reduction of Δ²⁴⁽²⁸⁾ catalyzed by ERG4. By comparing the substrate structures for enzymes involved in the steroid synthesis pathways and the reaction chiral selectivity of various plants and microorganisms, it is found that replacing the ERG4 with a sterol Δ²⁴⁽²⁸⁾ reductase DWF1 from the BL synthesis pathway of plant is most likely to enable yeast to switch from ergosterol synthesis to 24-epi-ergosterol synthesis. In addition, due to a large structural difference in natural substrates for the above two enzymes, DWF1 needs to be modified to improve its catalytic activity for non-natural substrates. Secondly, a metabolic flux towards the 24-epi-ergosterol in yeast is improved by manipulating the sterol homeostasis, maintaining a new balance between sterol acylation and sterol ester hydrolysis. Finally, the expression levels of genes involved in the synthetic pathway are further regulated to achieve a satisfactory yield of the 24-epi-ergosterol.

### SUMMARY

To overcome the deficiencies in the prior art, an objective of the present disclosure is to provide a fungus synthesizing 24-epi-ergosterol, a construction method therefor and use thereof.

In order to achieve the above objective, the present disclosure adopts the following technical solutions:

The present disclosure first provides a method for constructing a fungus synthesizing 24-epi-ergosterol, including recombinantly expressing a sterol Δ²⁴⁽²⁸⁾ reductase DWF1 gene or a mutant gene thereof into an ergosterol-producing fungus to obtain the fungus synthesizing 24-epi-ergosterol.

In some embodiments, the ergosterol-producing fungus is an ergosterol-producing eukaryotic microorganism or an ERG4 gene-knockout mutant thereof; preferably a eukaryotic microorganism selected from the group consisting of families Saccharomycesceae, Sclerotiniaceae, Cladosporiaceae, Hypocreaceae, Trichocomaceae, Aspergillaceae and Tricholomataceae; or an ERG4 gene-knockout mutant of the above eukaryotic microorganism; and particularly preferably an ERG4 gene-knockout Saccharomyces cerevisiae.

In some embodiments, a preparation process of the DWF1 mutant gene includes the following steps:
conducting error-prone PCR using a plant-derived sterol Δ²⁴⁽²⁸⁾ reductase DWF1 gene as a template in the presence of 0.02 mM to 0.12 mM of Mn²⁺ to create a DWF1 mutant library;
co-transforming the DWF1 mutant library with a linearized plasmid pRS42H into an ERG4-knockout ergosterol-producing fungal strain, and coating an obtained fungal strain on a solid culture medium containing 50 μg/mL to 200 μg/mL of Hyg B; selecting an obtained grown strain into a culture medium containing 50 μg/mL to 200 μg/mL of Hyg B and an ERG4-knockout strain growth inhibitor to allow cultivation;
selecting an obtained growing strain and inoculating the growing strain into a culture medium containing 50 μg/mL to 200 μg/mL of Hyg B to allow cultivation, conducting liquid chromatography analysis on obtained culture to obtain the DWF1 mutant gene with an improved catalytic activity.

Further, the ERG4-knockout strain growth inhibitor is one or more selected from the group consisting of 0.01 wt % to 0.05 wt % of sodium dodecyl sulfate (SDS), 2 μg/mL to 20 μg/mL of nystatin, and 10 μg/mL to 100 μg/mL of fluconazole.

Further, the DWF1 gene is derived from a brassinosteroids-producing plant; preferably a plant selected from the group consisting of families Lamiaceae, Cruciferae, and Moraceae; more preferably a plant with a relatively high brassinosteroids content; and particularly preferably Ajuga reptans (Ar).

In some embodiments, the construction method further includes: integrating an expression cassette sequence of ARE2, YEH1, and YEH2 set forth in SEQ ID NO: 7 at 16Ty3 site into the genome of the constructed strain by CRISPR/Cas9 editing, thereby overexpressing ARE2, YEH1, and YEH2 genes to enhance a homeostasis between esterification storage and hydrolysis release of sterols.

In some embodiments, the construction method further includes: integrating a sequence set forth in SEQ ID NO: 8 at the promoter of ACC1 gene into the constructed strain by CRISPR/Cas9 editing to take the place of the promoter of ACC1 gene and overexpress DWF1 gene or a mutant gene thereof, and integrating a sequence set forth in SEQ ID NO: 9 of a constructed ERG5 expression cassette at 15Ty2 site by the CRISPR/Cas9 editing to overexpress an ERG5 gene, thereby enhancing synthesis of the 24-epi-ergosterol while increasing a proportion of the 24-epi-ergosterol in the sterols. Further, the DWF1 gene encodes an amino acid sequence set forth in SEQ ID NO: 1.

The DWF1 mutant gene encodes an amino acid sequence set forth in any one selected from the group consisting of:
(1) an amino acid sequence set forth in SEQ ID NO: 2 in the sequence listing;
(2) an amino acid sequence set forth in SEQ ID NO: 3 in the sequence listing;
(3) an amino acid sequence set forth in SEQ ID NO: 4 in the sequence listing;
(4) an amino acid sequence set forth in SEQ ID NO: 5 in the sequence listing; and
(5) an amino acid sequence set forth in SEQ ID NO: 6 in the sequence listing; where

The sterol Δ²⁴⁽²⁸⁾ reductase having the amino acid sequence set forth in SEQ ID NO: 2 in the sequence listing is named ArV143G, and its valine at position 143 is mutated to glycine relative to the wild type. The sterol Δ²⁴⁽²⁸⁾ reductase having the amino acid sequence set forth in SEQ ID NO: 3 in the sequence listing is named ArM235T, and its methionine at position 235 is mutated to threonine relative to the wild type. The sterol Δ²⁴⁽²⁸⁾ reductase having the amino acid sequence set forth in SEQ ID NO: 4 in the sequence listing is named ArS306P, and its serine at position 306 is mutated to proline relative to the wild type. The sterol Δ²⁴⁽²⁸⁾ reductase having the amino acid sequence set forth in SEQ ID NO: 5 in the sequence listing is named ArY338H, and its tyrosine at position 338 is mutated to histidine relative to the wild type. The sterol Δ²⁴⁽²⁸⁾ reductase having the amino acid sequence set forth in SEQ ID NO: 6 in the sequence listing is named Ar207, its valine at position 143 is mutated to glycine, its serine at position 306 is mutated to proline, and its tyrosine at position 338 is mutated to histidine relative to the wild type.

Further, the plant-derived sterol Δ²⁴⁽²⁸⁾ reductase DWF 1 gene is derived from Arabidopsis thaliana (At), Ajuga reptans (Ar), Brassica rapa (Br), or Cannabis sativa (Cs).

Further, the plasmid is a pRS42H plasmid.

Further, the ergosterol-producing fungus is Saccharomyces cerevisiae.

The present disclosure further provides a fungus synthesizing 24-epi-ergosterol, where the fungus is constructed by the above construction method.

The present disclosure further provides use of the fungus synthesizing 24-epi-ergosterol in production of the 24-epi-ergosterol.

Compared with the prior art, the present disclosure has the following advantages:

The present disclosure provides a construction method of a fungus synthesizing 24-epi-ergosterol, where the obtained fungus may have an increased yield of the 24-epi-ergosterol. The present disclosure further proposes a further optimized construction method, such that the obtained fungus enhances a synthesis ability of the 24-epi-ergosterol while increasing a proportion of the 24-epi-ergosterol in sterols. The present disclosure establishes a high-throughput screening method for the catalytic activity modification of sterol Δ²⁴⁽²⁸⁾ reductase (DWF1). 4 mutation points are screened by the screening method: V143G, M235T, S306P, and Y338H. Moreover, an optimal mutation combination Ar207 (ArDWF1^{V143G/S306P/Y338H}) is obtained by combining mutations, such that a synthesis efficiency of 24-epi-ergosterol is significantly improved. Meanwhile, with the help of metabolic engineering, higher proportion production of 24-epi-ergosterol is also achieved, thus providing a desirable foundation for the industrial fermentation production of 24-epi-ergosterol. Moreover, the present disclosure provides a desirable precursor for the semi-synthesis of BL.

In the present disclosure, a novel sterol homeostasis is established by overexpressing sterol acylase ARE2 and hydrolases YEH1 and YEH2, to enhance sterol acylation and steryl ester hydrolysis simultaneously, increasing the sterol synthesis capacity in yeast, which is twice that of strain YQE231.

In the present disclosure, the promoters of Ar207 and ACC1 are replaced with stronger promoters by homologous recombination. At the same time, ERG5 gene is overexpressed to increase the expression levels of Ar207, ACC1, and ERG5, which not only enhances the synthesis capacity of 24-epi-ergosterol, but also increases the proportion of 24-epi-ergosterol in sterols, thereby reducing the pressure of purification in the later stage.

In the present disclosure, an engineered strain YQE734 increases the yield of 24-epi-ergosterol to 2.76 g/L through high-density fermentation, which is 3.28 times higher than that of strain YQE231.

### BRIEF DESCRIPTION OF THE DRAWINGS

FIG. 1 shows the structures of BL, EBL, and HBL;

FIG. 2 shows the synthesis diagrams of EBL, HBL, and BL from ergosterol, stigmasterol, crinosterol, and 24-epi-ergosterol, respectively;

FIG. 3 shows a biosynthetic pathway of BL in plants;

FIG. 4 shows a synthetic pathway of 24-epi-ergosterol in Saccharomyces cerevisiae;

FIG. 5 shows the growth curves of an industrial strain CICC4746 and a model strain BY4741;

FIG. 6 shows a standard curve of the ergosterol;

FIG. 7 shows the growth inhibition of strains with different ergosterol/24-epi-ergosterol yields at different SDS concentrations to determine an SDS concentration suitable for high-throughput screening;

FIG. 8 shows the relative enzyme activities of four mutants (ArV143G, ArM235T, ArS306P, and ArY338H) obtained by high-throughput screening in the form of plasmids in YQE101;

FIG. 9 shows the yield of 24-epi-ergosterol after the combined mutations are integrated into a genome of the industrial strain;

FIG. 10 shows a map of the plasmid (pRS42H);

FIG. 11 shows a map of the plasmid pEB-Y1A2Y2;

FIG. 12 shows a map of the plasmid pRS42H-P_{GAL1}-Ar207;

FIG. 13 shows a map of the plasmid pRS42H-P_{CIT2}-ERG5;

FIG. 14 shows effects of overexpression of sterol acylase ARE2 and hydrolases YEH1 and YEH2, replacement of promoters of Ar207 and ACC1, and overexpression of ERG5 on the yield of 24-epi-ergosterol and precursors thereof; where YQE231 represents an original strain, serving as a control group; YQE717 represents a strain in which sterol acylase ARE2 and hydrolases YEH1 and YEH2 are overexpressed; YQE729 represents a strain derived from YQE717 in which the promoters of Ar207 and ACC1 are replaced; and YQE734 represents a strain derived from YQE729 in which ERG5 is overexpressed; and

FIG. 15 is a diagram showing a high-density fermentation of the YQE734.

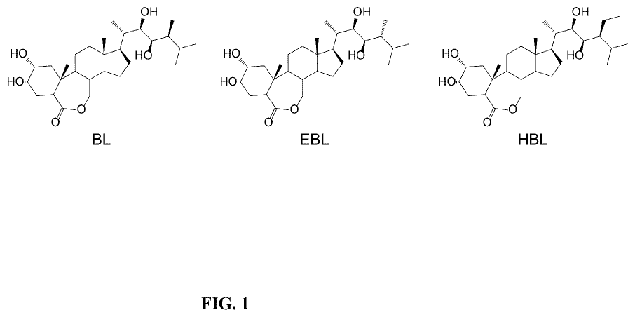

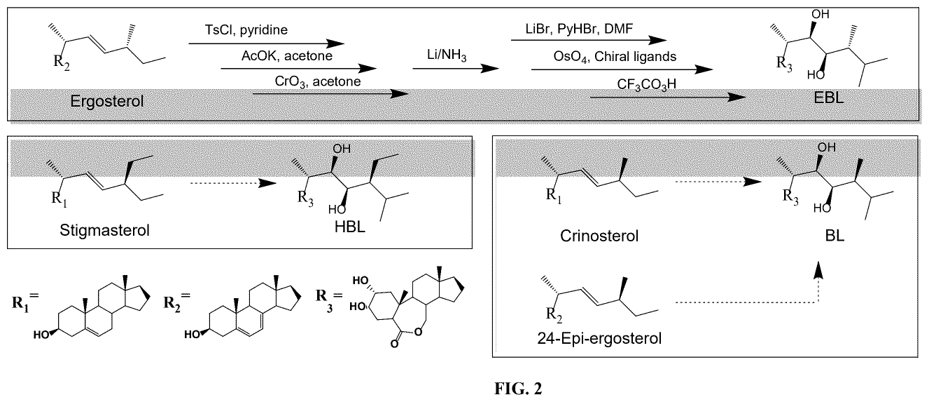

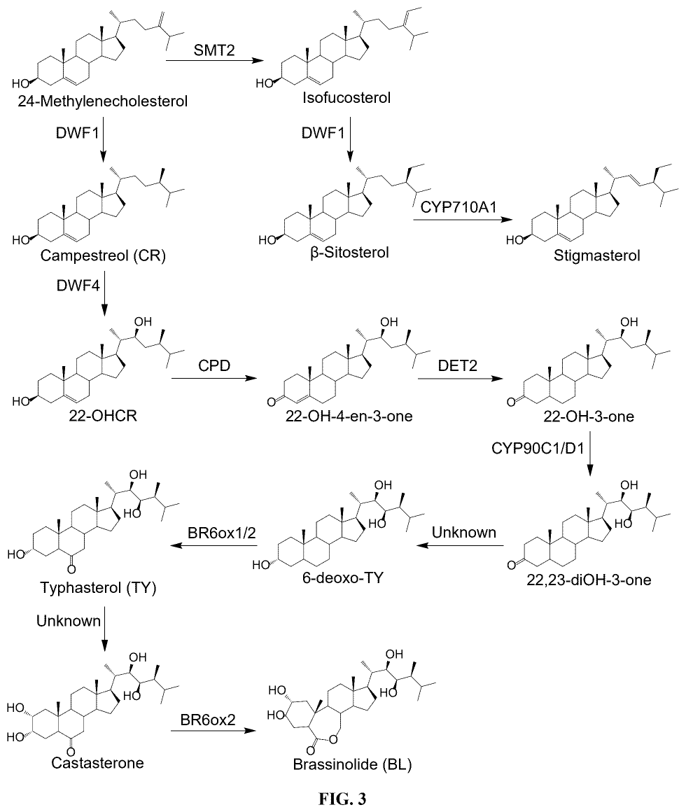

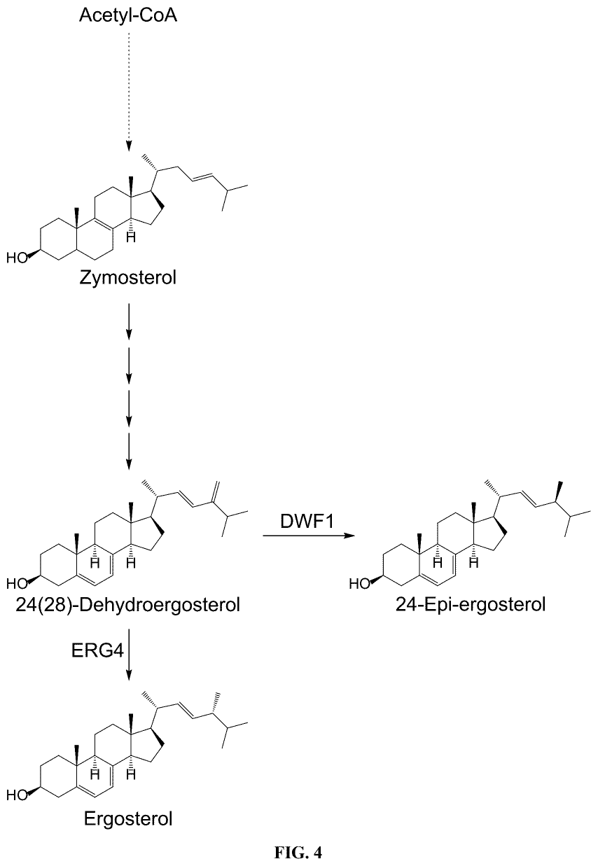

### DETAILED DESCRIPTION OF THE EMBODIMENTS

The present invention is described in further detail below with reference to specific implementations. The technical features in each implementation of the present invention can be correspondingly combined on the premise that they are not conflicted.

In the present disclosure, the culture media used are as follows:
(1) YPD culture medium (1% yeast extract, 2% peptone, and 2% glucose), 2% agar powder was added when prepare a solid medium, sterilized at 115° C., for activation and pre-culture of Saccharomyces cerevisiae.
(2) YPD (HygB/G418) culture medium (1% yeast extract, 2% peptone, 2% glucose, 100 μg/mL HygB, and 200 μg/mL G418), 2% agar powder was added when prepare a solid medium and sterilized at 115° C., for screening KanMX marker.
(3) LB culture medium (1% sodium chloride, 1% tryptone, and 0.5% yeast extract), 2% agar powder was added when prepare a solid medium, sterilized at 121°° C., for activation and pre-culture of Escherichia coli.
(4) LB (Amp) culture medium (1% sodium chloride, 1% tryptone, 0.5% yeast extract, and 100 μg/mL ampicillin), 2% agar powder was added when prepare a solid medium and sterilized at 121° C., for culturing Escherichia coli containing a plasmid.

In the present disclosure, a strain object targeted by the construction method may be any fungus that produces ergosterol. The ergosterol is a component of fungal cell membrane in nature, and almost all of the fungi produce ergosterol. Typically, Saccharomyces cerevisiae may be used. In the examples, an industrial Saccharomyces cerevisiae strain CICC1746 is used as a main experimental subject, however, other Saccharomyces cerevisiae strains or ergosterol-producing fungi may also be selected. The following industrial Saccharomyces cerevisiae strain (strain number: CICC1746) was purchased from China Center of Industrial Culture Collection (CICC), and the model strain BY4741 was purchased from American Type Culture Collection (ATCC).

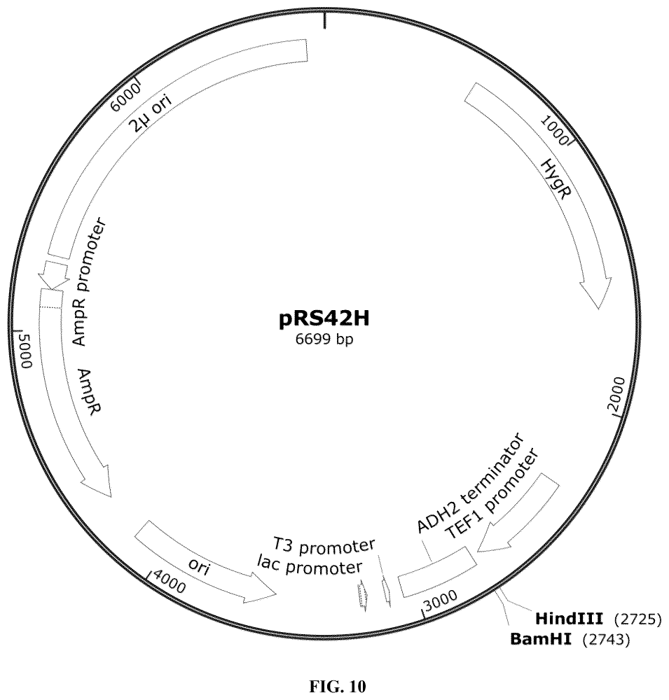

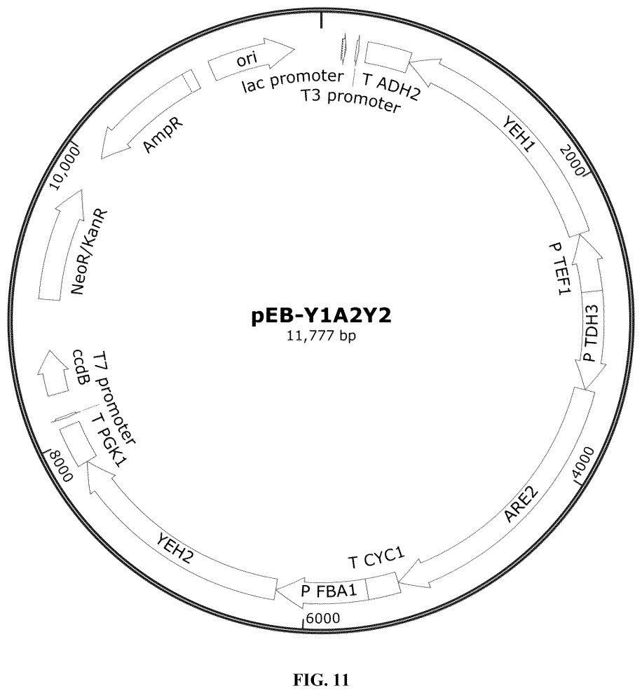

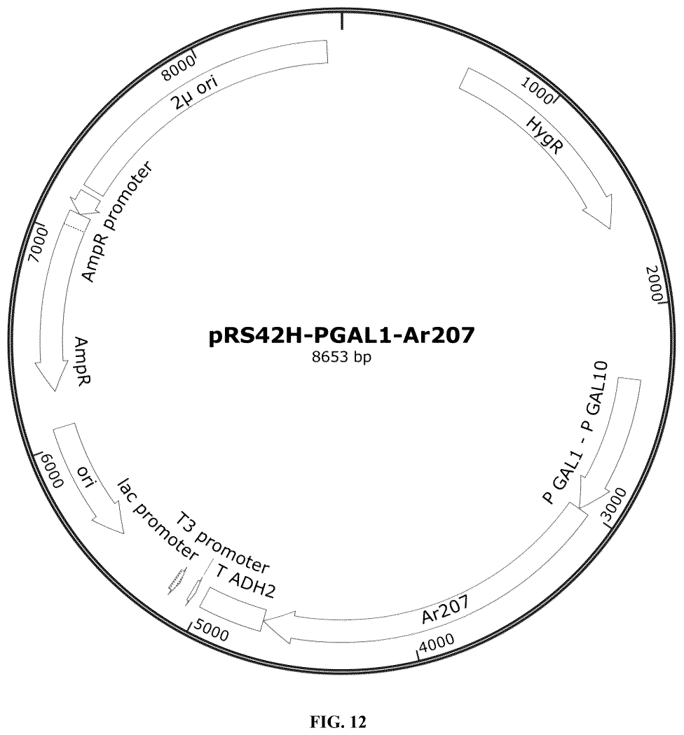

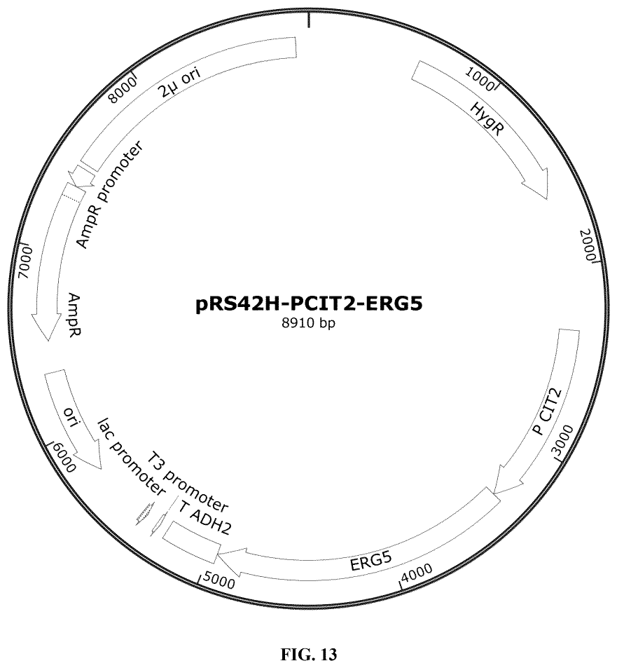

In the present disclosure, a map of the plasmid pRS42H used is shown in FIG. 10;
a map of the plasmid pEB-Y1A2Y2 used is shown in FIG. 11;
a map of the plasmid pRS42H-P_{GAL1}-Ar207 used is shown in FIG. 12; and
a map of the plasmid pRS42H-P_{CIT2}-ERG5 used is shown in FIG. 13.

### Example 1: Constructing ERG4-Knockout Yeast Strains YQE101 and YQE102

Using the genome of industrial Saccharomyces cerevisiae strain CICC1746 as a template, PCR amplification was conducted with primers erg4Δ-U-F (SEQ ID NO: 10)/erg4Δ-U-R (SEQ ID NO: 11) to obtain a 449 bp homology arm fragment erg4Δ-U upstream of the ERG4 site, while PCR amplification was conducted with primers erg4Δ-D-F (SEQ ID NO: 12)/erg4Δ-D-R (SEQ ID NO: 13) to obtain a 446 bp homology arm fragment erg4Δ-D downstream of the ERG4 site. The 2 amplified fragments were ligated by overlap extension PCR to obtain a Kerg4 fragment. The Kerg4 fragment was transformed into a model strain BY4741 and an industrial Saccharomyces cerevisiae strain CICC1746 by lithium acetate/PEG3350 chemical transformation. Using a CRISPR-Cas9 gene editing tool, DNA was cleaved to produce double-strand breaks at the ERG4 site guided by ERG4-gRNA (SEQ ID NO: 59), and positive clones by transformation were selected in YPD medium containing 100 μg/mL Hyg B and 200 μg/mL G418 to obtain the ERG4-knockout strains YQE101 (BY 4741; erg4Δ) and YQE 102 (CICC1746; erg4 Δ).

### Example 2: Constructing 24-epi-ergosterol-Producing Saccharomyces cerevisiae Strain YQE224

Using the ArDWF1 plasmid containing sterol Δ²⁴⁽²⁸⁾ reductase from Ajuga reptans synthesized by Sangon Biotech (Shanghai) Co., Ltd. as a template, PCR amplification was conducted using primers VArDWF1-F (SEQ ID NO: 16)/VArDWF1-R (SEQ ID NO: 17) to obtain an ArDWF1 fragment. The ArDWF1 fragment was ligated to a linearized pRS42H plasmid by seamless cloning to obtain a gene expression plasmid pRS42H-ArDWF1. Using the gene expression plasmid pRS42H-ArDWF1 as a template, PCR amplification was conducted using primers Dor.-16Ty1-P_{TEF1} (SEQ ID NO: 14)/Dor.-16Ty1-P_{ADH2} (SEQ ID NO: 15) to obtain an ArDWF1 expression cassette. The ArDWF1 expression cassette was transformed into YQE102 by lithium acetate/PEG3350 chemical transformation. Using a CRISPR-Cas9 gene editing tool, DNA was cleaved at the 16Ty1 site to produce double-strand breaks under the guidance of 16Ty1-gRNA (SEQ ID NO: 60), to obtain YQE224 producing 24-epi-ergosterol. Through YPD shake flask fermentation verification and liquid chromatography, a yield of the 24-epi-ergosterol reached 13.97 mg/L.

### Example 3: Establishing a High-Throughput Screening Method for the ArDWF1 Catalytic Activity Modified Mutants

After knocking out the ERG4 gene, yeast cells, such as YQE101 and YQE102, would accumulate 24 28)-dehydroergosterol as a final product of the sterol synthesis pathway due to the lack of a reductase for the 24(28) double bond. Due to the lack of ergosterol, yeast cells might show certain growth defects in the growth environment, manifested as reduced tolerance to antibiotics, metal ions, and SDS. And 24-epi-ergosterol differed from ergosterol only in its 24-C chirality in structure. Therefore, a high-throughput screening method could be established by coupling DWF1 activity to cell growth under SDS and HygB stressed conditions. To ensure sensitivity and accuracy during the screening, the factors that might affect in the experiment were first optimized.

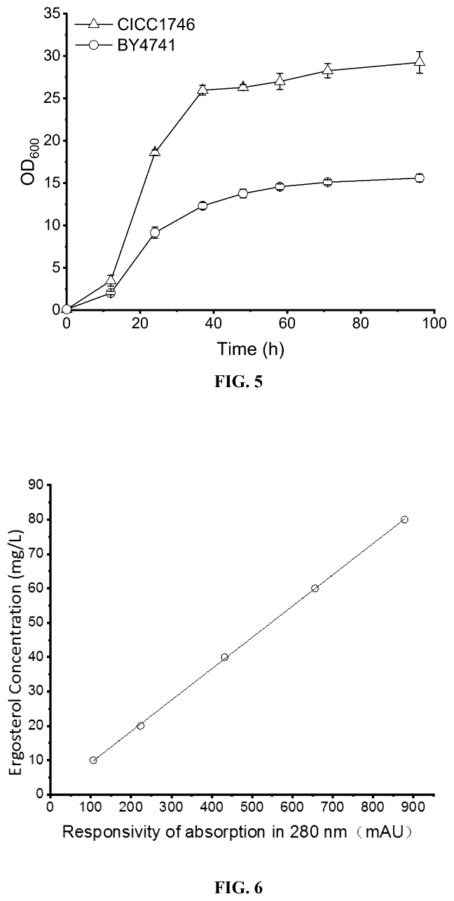

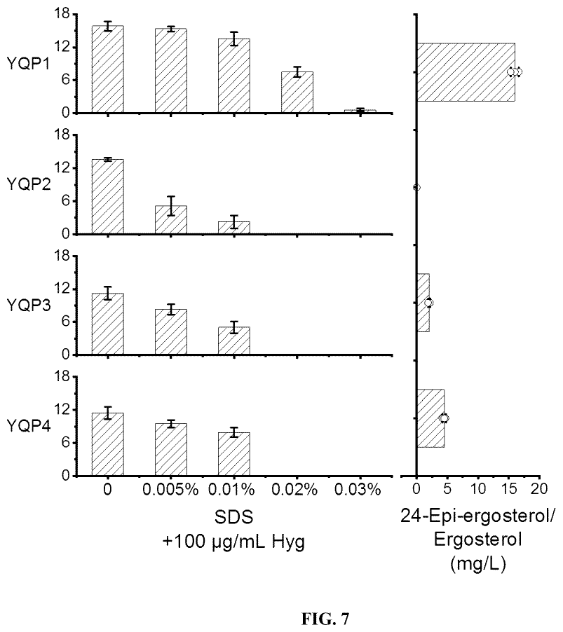

First, selection of a strain. Since there were obvious differences in growth between the industrial strain CICC1746 and the model strain BY 4741 (FIG. 5), the ERG 4-knockout strain YQE101 originated from BY4741 was selected as a host strain for high-throughput screening in order to facilitate the establishment of the screening method. Secondly, in order to determine the optimal SDS concentration for screening, the growth inhibition under different SDS concentrations of four strains producing different amounts of ergosterol/24-epi-ergosterol were measured (YQP1: BY4741 with plasmid pRS42H; YQP2: YQE101 with plasmid pRS42H; YQP3: YQE101 with plasmid pRS42H-AtDWF1 derived from Arabidopsis thaliana; YQP4: YQE101 with plasmid pRS42H-ArDWF1), as shown in FIG. 6 and FIG. 7, and YPD containing 100 μg/mL HygB+0.025% SDS was selected for high-throughput screening.

### Example 4: Constructing and High-Throughput Screening of Mutant Library

Using the ArDWF1 coding gene as a template, error-prone PCR was conducted with primers Ep-ArDWF1-F (SEQ ID NO: 18)/Ep-ArDWF1-R (SEQ ID NO: 19) in the presence of 0.07 mM Mn²⁺ to construct an ArDWF1 mutant library. Further, the plasmid pRS42H was linearized using restriction endonucleases HindIII and BamHI, and the ArDWF1 mutant library and the linearized pRS42H were co-transformed into YQE101 at a mol ratio of 6:1 using a lithium acetate/PEG3350 chemical transformation. The ArDWF1 mutant library and the linearized pRS42H were ligated to form complete plasmids by the homologous recombinase of the ERG4-knockout strain YQE101, and positive clones were screened on a plate containing 100 μg/mL hygromycin B (Hyg B) and 0.01% sodium dodecyl sulfate (SDS) to obtain a Saccharomyces cerevisiae strain with complete plasmid harboring successfully an ArDWF1 mutant. Then, the resulting strain was selected and inoculated into a 24-well plate with 2 mL YPD (including 2% w/v glucose, 1% w/v yeast extract, and 2% w/v peptone) containing 100 μg/mL Hyg B and 0.025% SDS for 3 days. The culture solution with a significantly increased yeast concentration (OD₆₀₀) was selected and inoculated into a test tube with 5 mL YPD containing 100 μg/mL Hyg B, cultured for 4 days, and re-screening was conducted by liquid chromatography. Since the ArDWF1 mutant was expressed in the model strain in the form of a free plasmid, antibiotics needed to be added in medium to maintain the plasmid, which would have an adverse effect on the growth of the fungus.

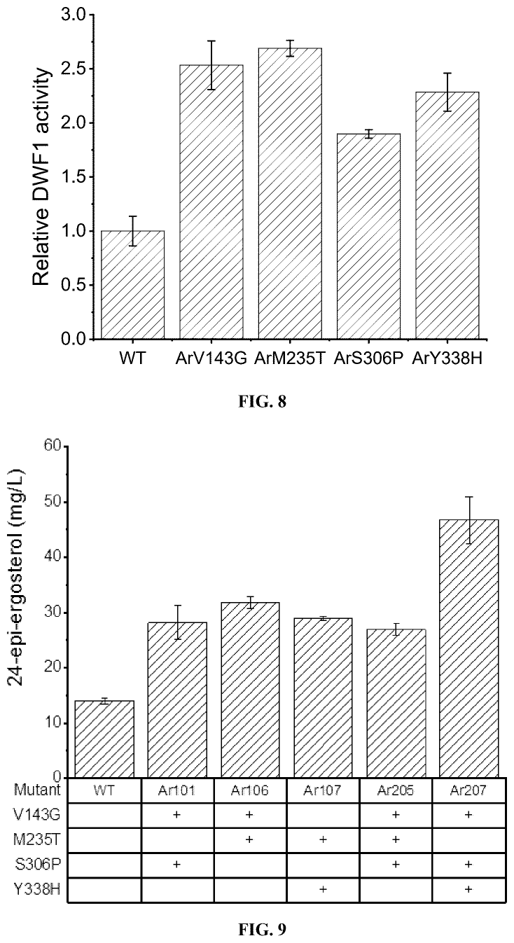

As a result, a relative DWF1 activity defined as a ratio of 24-epi-ergosterol to 24(28)-dehydroergosterol was used as the main comparison object. As shown in FIG. 8, 4 ArDWF1 mutants with relative DWF1 activity significantly improved compared with that of the wild-type ArDWF1 (ArV143G, ArM235T, ArS306P, and ArY338H) were finally obtained.

### Example 5: Combinatorial Mutation of ArDWF1

Using the recombinant plasmid pRS42H-ArDWF1 as a template, the 4 mutation points V143G, M235T, S306P, and Y338H, which significantly improved the DWF1 activity compared with that of the wild-type ArDWF1, were subjected to combined mutation. The primers corresponding to V143G were Ar-V143G-F (SEQ ID NO: 20)/Ar-V143G-R (SEQ ID NO: 21), the primers corresponding to M235T were Ar-M235T-F (SEQ ID NO: 22)/Ar-M235T-R (SEQ ID NO: 23), the primers corresponding to S306P were Ar-S306P-F (SEQ ID NO: 24)/Ar-S306P-R (SEQ ID NO: 25), and the primers corresponding to Y338H were Ar-Y338H-F (SEQ ID NO: 26) /Ar-Y338H-R (SEQ ID NO: 27). Using the plasmid after combined mutation as a template, PCR amplification was conducted using primers Dor.-16Ty1-P_{TEF1} (SEQ ID NO: 14)/Dor.-16Ty1-P_{ADH2} (SEQ ID NO: 15) with a 50 bp homology arm each to obtain an ArDWF1 combined mutation expression cassette. Through the lithium acetate/PEG3350 chemical transformation, the combined mutation expression cassette was transformed into the ERG4-knockout industrial yeast YQE102, which was then integrated into a genome of the industrial yeast with the help of a CRISPR-cas9 gene editing tool to obtain a strain YQE231. As shown in FIG. 9, an optimal combination mutant Ar207 (V143G/S306P/Y338H), with a 24-epi-ergosterol yield of 46.72 mg/L was determined by YPD shake flask fermentation verification and liquid phase detection, which was 334.4% higher than that of the wild-type ArDWF1.

### Example 6: Fermentation, Post-Treatment, and Liquid Chromatography Detection

The successfully edited transformants were each streaked onto YPD plates and cultured at 30° C. for 48 h. Single clones were selected and inoculated into 5 mL YPD liquid culture medium and cultured at 30° C. and 220 rpm for 96 h. After the culture was completed, 500 μL of a fermentation broth was collected, centrifuged to remove supernatant, and vortexed thoroughly with 600 μL of ethanol-KOH solution (25% [w/v] KOH added in 50% ethanol). A resulting product was saponified in a boiling water bath for 1 h, cooled to room temperature, and vortexed thoroughly with 400 μL of water and 800 μL of petroleum ether. 500 μL of the upper petroleum ether extract from a resulting product was vacuum-dried, redissolved with 500 μL of anhydrous ethanol, and filtered through a 0.22 μm PVDF filter into a cannula in a liquid phase detection bottle to obtain a test sample. The content of metabolites in the sample were analyzed by Agilent 1160 high-performance liquid chromatography (HPLC), where the chromatographic column was Thermo C-18 column (ODS Hypersil, 4.6×250 mm, 5 μm), the detection wavelength was 280 nm, the analytical column was maintained at a constant temperature of 30° C., the mobile phase was: methanol:acetonitrile=80:20, and a standard curve was plotted using a solution prepared with ergosterol standard to allow quantitative analysis.

### Example 7: A Method for Constructing a Strain Overexpressing ARE2, YEH1, and YEH2

A construction method of plasmid pEB-Y1A2Y2 for constructing the strain overexpressing ARE2, YEH1, and YEH2 includes the following steps:

Using plasmid pRS42H as a template, PCR amplification was conducted with primers pEB-T_{ADH2}-R (SEQ ID NO: 28) and pEB-P_{TEF1}-F (SEQ ID NO: 29) to obtain a P_{TEF1}-HindIII-BamHI-T_{ADH2} fragment. Using the genome of Saccharomyces cerevisiae strain CICC1746 as a template, PCR amplification was conducted with primers pEB-P_{TDH3}-F (SEQ ID NO: 30) and pEB-P_{TDH3}-R (SEQ ID NO: 31) to obtain P_{TDH3}-Xhol-AfIII fragment, primers pEB-T_{CYC1}-F (SEQ ID NO: 32) and pEB-T_{CYC1}-R (SEQ ID NO: 33) to obtain Xhol-AfIII-T_{CYC1} fragment, primers pEB-P_{FBA1}-F (SEQ ID NO: 34) and pEB-P_{FBA1}-R (SEQ ID NO: 35) to obtain P_{FBA1}-EcoRI-BInI fragment, and primers pEB-T_{PGK1}-F (SEQ ID NO: 36) and pEB-T_{PGK1}-R (SEQ ID NO: 64) to obtain EcoRI-BInI-T_{PGKI} fragment. There were homologous sequences between the fragments, and the fragments were fused by overlap extension PCR and constructed using a pEASY®-Blunt Simple Cloning Vectors K it to obtain pEB.

Using the genome of Saccharomyces cerevisiae strain CICC1746 as a template, PCR amplification was conducted with primers VYEH1-F (SEQ ID NO: 37) and VYEH1-R (SEQ ID NO: 38) to obtain YEH1 fragment, primers P_{TDH3}-ARE2 (SEQ ID NO: 39) and ARE2-T_{CYC1} (SEQ ID NO: 40) to obtain ARE2 fragment, and primers P_{PBAI}-YEH2 (SEQ ID NO: 41) and YEH2-T_{PGK1} (SEQ ID NO: 42) to obtain YEH2 fragment. The pEB-Y1A2Y2 was constructed by enzyme digestion and recombination.

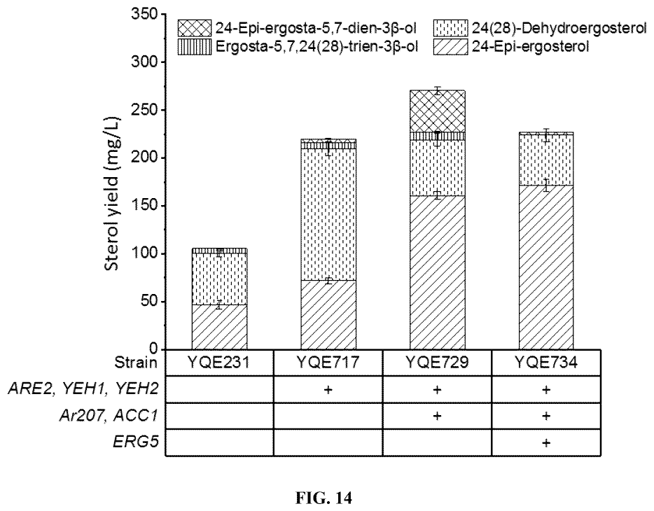

A method for constructing the strain overexpressing ARE2, YEH1, and YEH2 includes the following steps:
(1) PCR amplification was conducted using primers Dor.-16Ty3-1-T_{ADH2} (SEQ ID NO: 43)/Dor.-16Ty3-1-T_{PGK1} (SEQ ID NO: 44) with a 50 bp homology arm each to obtain an ARE2, YEH1, and YEH2 expression cassette.
(2) The ARE2, YEH1, and YEH2 expression cassette was transformed into Saccharomyces cerevisiae strains Y QE231 and Y QE 102 using lithium acetate/PEG 3350 chemical transformation.
(3) Using CRISPR-Cas9 gene editing tool under the guidance of 16Ty3-gRNA (SEQ ID NO: 61), DNA was cleaved at the 16T y3 site to produce double-strand breaks, and the expression cassette was integrated into the genome of Saccharomyces cerevisiae. The resulted strains were coated on a HygB/G418 plate for positive clone strains selection.
(4) Single colonies were selected from each plate, cultured overnight in shake flasks, and the genome of the single colonies were extracted for PCR to verify whether the ARE2, YEH1, and YEH2 expression cassette was integrated into the 16Ty3 site of genome.
(5) Positive clones were selected based on the PCR verification results, and YQE717 (YQE231; 16 Ty3::T_{ADH2}-YEH1-P_{TEF1}-P_{TDH3}-ARE2-T_{CYC1}-P_{FBA1}-YEH2-T_{PGK1}) and YQE103 (YQE102; 16Ty3::T_{ADH2}-YEH1-P_{TEF1}-P _{TDH3}-ARE2-T_{CYC1}-P_{FBA1}-YEH2-T_{PGK1}) were constructed.
(6) Fermentation, post-treatment, and liquid chromatography detection were conducted. As shown in FIG. 14, YQE717 had a significant improvement in total late sterol yield and 24-epi-ergosterol yield. At the shake flask level, the total late sterol yield and 24-epi-ergosterol yield reached 220.07 mg/L and 71.04 mg/L, which were 2.09 times and 1.53 times that of strain Y QE231, respectively.

### Example 8: Determination of Transcription Levels of ARE2, YEHI, and YEH2

The original strain (YQE231) and the overexpression strain (YQE717) were cultured in liquid YPD medium. During the shake flask fermentation, 1 mL of a fermentation broth was taken at 12 h, 24 h, 36 h, 48 h, and 72 h separately.

The total RNA of each sample was extracted using the RNA extraction kit TRIzol (Invitrogen). The genomic DNA was removed and the total RNA was reverse transcribed to obtain cDNA using a reverse transcription kit (TOYOBO). Specific operations were conducted with reference to the kit instructions.

Using ACT1 gene as a reference gene, real-time fluorescence quantitative PCR was conducted on ARE2, YEH1, and YEH2 to obtain amplification curves and CT values were recorded. The relative transcription levels of ARE2, YEHI, and YEH2 were calculated using the 2^{-ΔΔCT} method.

### Example 9: Strain Replacing the Promoter of ACC1 and Ar207

A construction method of the plasmid pRS42H-P_{GAL1}-Ar207 for replacing the promoter of ACC1 and Ar207 includes the following steps:

Using pRS42H-Ar207 as a template, primers R-pRS42H-207-F (SEQ ID NO: 45) and R-pRS42H-207-R (SEQ ID NO: 46) were used for amplification to obtain a promoter-removed fragment. Using the genome of CICC1746 as a template, primers P_{GAL10-GAL1}-F (SEQ ID NO: 47) and P_{GAL10-GAL1}-R (SEQ ID NO: 48) were used to amplify a PGAL10-P_{GAL1} fragment. A pRS42H-P_{GAL1}-Ar207 was obtained by recombination construction.

A method for constructing a strain replacing the promoter of ACC1 and Ar207 included the following steps:
(1) A P_{GAL10}-P_{GAL1}-Ar207 expression cassette was obtained by PCR amplification using primers Dor.-P_{ACC1}-P_{GAL10} (SEQ ID NO: 49)/Dor.-P_{ACC1}-T_{ADH2} (SEQ ID NO: 50) each with a 50 bp homology arm.
(2) The above P_{GAL10}-P_{GAL1}-Ar207 expression cassette was transformed into Saccharomyces cerevisiae strain YQE103 by lithium acetate/PEG3350 chemical transformation.
(3) Using CRISPR-Cas9 gene editing tool under the guidance of P_{ACC1}-gRNA (SEQ ID NO: 62), the ACC1 promoter P_{ACC1} site was cleaved to form double-strand breaks, and the P_{GAL10}-P_{GAL1}-Ar207 expression cassette was integrated into this site. The resulted strain was coated on HygB/G418plates for positive clone strains selection.
(4) A single colony was selected from the plate, cultured overnight in a shake flask, and a genome of the single colony was extracted for PCR to verify whether the P_{GAL10}-P_{GAL1}-Ar207 expression cassette was integrated into the P_{ACC1} site.
(5) Based on the PCR verification results, positive clones were selected and YQE729 was constructed.
(6) By fermentation, post-treatment, and liquid chromatography detection, as shown in FIG. 14, YQE729 had a significant improvement in 24-epi-ergosterol yield. At the shake flask level, the 24-epi-ergosterol yield reached 160.84 mg/L, which was 2.26 times that of YQE717.

### Example 10: A Method for Constructing an ERG5-Overexpressing Strain

A construction method of the plasmid pRS42H-P_{CIT2}-ERG5 for constructing an ERG5-overexpressing strain includes the following steps:

Using pRS42H-Ar207 as a template, amplification was conducted with primers R-pRS42H-207-F and R-pRS42H-207-R to obtain a promoter-removed fragment. Using the genome of CICC1746as a template, amplification was conducted with primers P_{CIT2}-F (SEQ ID NO: 51) and P_{CIT2}-R (SEQ ID NO: 52) to obtain a P_{CIT2} fragment. A pRS42H-P_{CIT2}-Ar207 was constructed by gene recombination.

Using pRS42H-P_{CIT2}-Ar207 as a template, amplification was conducted with primers R-P_{CIT2}-R (SEQ ID NO: 53) and R-T_{ADH2}-F (SEQ ID NO: 54) to obtain a backbone fragment. Using the genome of CICC1746 as a template, amplification was conducted with primers ERG5-F (SEQ ID NO: 55) and ERG5-R (SEQ ID NO: 56) to obtain a fragment of ERG5. A pRS42H-P_{CIT2}-ERG5 was constructed by gene recombination.

A method for constructing an ERG 5-overexpressing strain includes the following steps:
(1) An ERG5 expression cassette was obtained by PCR amplification using primers Dor.-15Ty2-F (SEQ ID NO: 57)/Dor.-15Ty2-R (SEQ ID NO: 58) with a 50 bp homology arm each.
(2) The above ERG5 expression cassette was transformed into Saccharomyces cerevisiae strain using lithium acetate/PEG 3350 chemical transformation.
(3) Using CRISPR-Cas9 gene editing tool under the guidance of 15Ty2-gRNA (SEQ ID NO: 63), DNA was cleaved at the 15Ty2 site to form double-strand breaks, and the expression cassette was integrated into the genome of strain YQE729. The resulted strain was coated on HygB/G418 plates to select positive clone strains.
(4) A single colony was selected from the plate, cultured overnight in a shake flask, and a genome of the single colony was extracted for PCR to verify whether the ERG5 expression cassette was integrated into the 15Ty2 site on the genome.
(5) Based on the PCR verification results, positive clones were screened and YQE734 was constructed.
(6) By fermentation, post-treatment and liquid chromatography detection, as shown in FIG. 14, YQE734 has a significantly increased proportion of 24-epi-ergosterol in total sterols. At the shake flask level, the proportion of 24-epi-ergosterol increased to 75.44% based on the yield of 171.31 mg/L, which was 1.27 times to the portion in YQE729.

### Example 11: Fermentation Verification of Engineered Strains in Fermenter

A single colony was inoculated into 5 mL of YPD medium, cultured at 30° C. and 220 rpm for 24 h, and then transferred into two YPD shake flasks containing 50 mL of YPD medium at an inoculum volume of 2% (v/v). After culturing for 20 h, the culture broths in the two shake flasks were inoculated into a 2 L bioreactor (the ingredients of the culture medium in the fermenter include 10 g/L D-glucose, 10 g/L (NH₄)₂SO₄, 8 g/L KH₂PO₄, 3 g/L MgSO₄·0.72 g/L ZnSO₄·7H₂O, 10 mL/L trace metal solution, and 12 mL/L vitamin solution; the initial fermentation volume after inoculation was 1 L).

Fermenter parameter settings: temperature set at 30° C., ammonia was supplemented to maintain the pH value at 5.0, and dissolved oxygen was maintained at >25% saturation by adjusting the agitation rate (300 rpm to 950 rpm) and airflow rate (1 vvm to 3 vvm). After the carbon source in the initial culture medium was used up, a feeding solution containing 500 g/L glucose and 12 mL/L vitamin solution was fed into the fermenter according to the pseudo-exponential feeding model; when the OD₆₀₀ value of the fungal cells in the fermenter reached 200, anhydrous ethanol was added at a rate of 6 mL/h until the fermentation ended. The feeding rate F_{S} during the pseudo-exponential feeding phase was determined by the following equation:

$${F}_{s} = ( \frac{μ}{{Y}_{X / S}} + m ) \cdot \frac{{X}_{0} {V}_{0}}{S} \cdot {e}^{μ t}$$

where X₀, V₀, and S represent the initial biomass density (gDCW/L), the initial culture volume (L), and the glucose concentration in the culture medium (g/L); Y_{X/S} represents the yield of cell biomass to glucose (gDCW/g glucose); μ represents the specific growth rate (h⁻¹); m represents the maintenance factor (g glucose/gDCW/h), and t represents the time after the start of feeding (h). The specific growth rate was set to 0.12 h⁻¹, Y_{X/S} was set to 0.5, and m was set to 0.05.

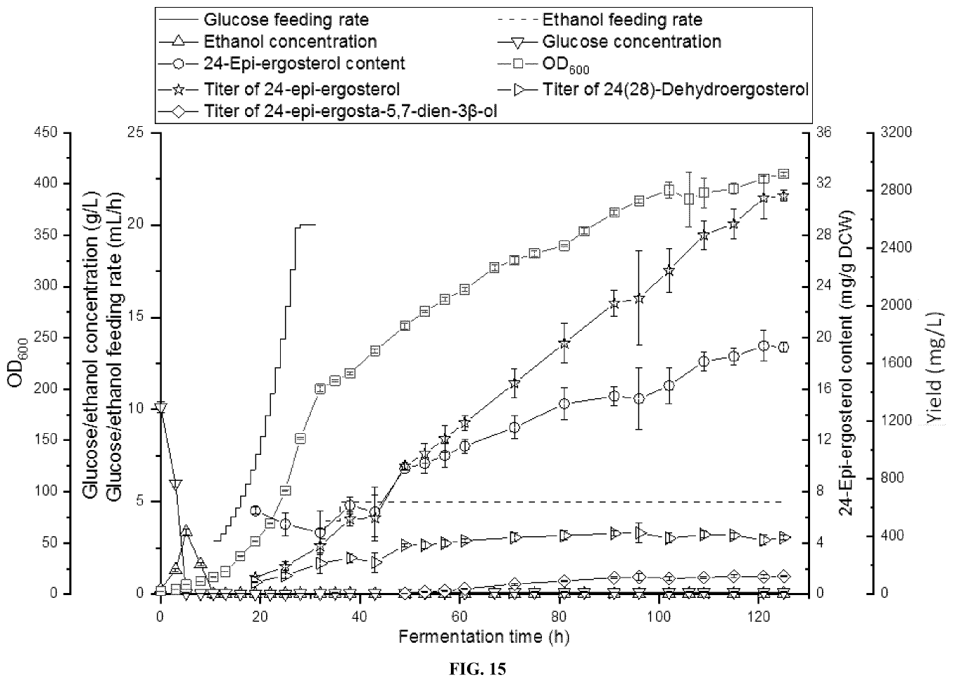

After the fermentation, the 24-epi-ergosterol yield of the YQE734 strain was detected by HPLC. The test results (FIG. 15) show that the strain YQE734 produced 2.76 g/L 24-epi-ergosterol in a 2 L fermenter. This study laid a desirable foundation for the industrial production of 24-epi-ergosterol by this strain.

Although the embodiments of the present disclosure have been illustrated and described above, it can be understood that the above embodiments are exemplary and cannot be construed as a limitation to the present disclosure. A person of ordinary skill in the art may make various changes, modifications, replacements and variations to the above embodiments without departing from the principle and spirit of the present disclosure.

What is claimed is:

1. An engineered strain synthesizing 24-epi-ergosterol, comprising a sterol Δ²⁴⁽²⁸⁾ reductase DWF1 or a DWF1 mutant.

2. The engineered strain according to claim 1, wherein the engineered strain is a eukaryotic microorganism.

3. The engineered strain according to claim 1, wherein the engineered strain is an ERG4 gene-knockout eukaryotic microorganism.

4. The engineered strain according to claim 3, wherein the engineered strain is an ERG4 gene-knockout eukaryotic microorganism carrying or overexpressing an ERG5 gene.

5. The engineered strain according to claim 3, wherein the eukaryotic microorganism is selected from the group consisting of families Saccharomycesceae, Sclerotiniaceae, Cladosporiaceae, Hypocreaceae, Trichocomaceae, Aspergillaceae and Tricholomataceae.

6. The engineered strain according to claim 1, wherein the eukaryotic microorganism is a 24 (28)-dehydroergosterol-synthesizing eukaryotic microorganism.

7. The engineered strain according to claim 1, wherein the DWF1 has the amino acid sequence set forth in SEQ ID NO: 1.

8. The engineered strain according to claim 1, wherein a gene of the DWF1 mutant was obtained by the following steps:
conducting error-prone PCR using a plant-derived sterol Δ²⁴⁽²⁸⁾ reductase DWF1 gene as a template under 0.02 mM to 0.12 mM of Mn²⁺;
create a DWF1 mutant library;
co-transforming the DWF1 mutant library with a linearized plasmid pRS42H into an ERG4-knockout strain;
selecting an obtained grown strain and inoculating the grown strain into a culture medium containing 50 μg/mL to 200 μg/mL of Hyg B and an ERG4-knockout strain growth inhibitor; and
selecting an obtained growing strain and inoculating the growing strain into a culture medium containing 50 μg/mL to 200 μg/mL of Hyg B to allow cultivation, subjecting obtained strain by the cultivation to saponification, extraction, and liquid chromatography analysis in sequence to screen out a strain with an improved enzyme activity; extracting a plasmid from the strain with an improved enzyme activity, and sequencing the plasmid to obtain a sequence for the DWF1 mutant with an improved catalytic activity.

9. The engineered strain according to claim 8, wherein the sterol Δ²⁴⁽²⁸⁾ reductase DWF1 gene is derived from a brassinosteroids-producing plant.

10. The engineered strain according to claim 9, wherein the plant is derived from families Lamiaceae, Cruciferae, or Moraceae.

11. The engineered strain according to claim 10, wherein the plant is Ajuga reptans.

12. The engineered strain according to claim 8, wherein the ERG4-knockout strain growth inhibitor is one or more selected from the group consisting of 0.01 wt % to 0.05 wt % of sodium dodecyl sulfate (SDS), 2 μg/mL to 20 μg/mL of nystatin, and 10 μg/mL to 100 μg/mL of fluconazole.

13. The engineered strain according to claim 8, wherein the DWF1 mutant is a mutant having one or more mutations selected from a to d below:
a, valine at position 143 of the DWF1 having the amino acid sequence set forth in SEQ ID NO: 1 mutated to glycine;
b, methionine at position 235 of the DWF1 having the amino acid sequence set forth in SEQ ID NO: 1 mutated to threonine;
c, serine at position 306 of the DWF1 having the amino acid sequence set forth in SEQ ID NO: 1 mutated to proline; and
d, tyrosine at position 338 of the DWF1 having the amino acid sequence set forth in SEQ ID NO: 1 mutated to histidine.

14. The engineered strain according to claim 8, wherein the DWF1 mutant is a mutant of the DWF1 having the amino acid sequence set forth in SEQ ID NO: 1 in which valine at position 143 is mutated to glycine, serine at position 306 is mutated to proline, and tyrosine at position 338 is mutated to histidine; and the DWF1 mutant has the amino acid sequence set forth in SEQ ID NO: 6.

15. The engineered strain according to claim 6, wherein an original strain of the Saccharomyces cerevisiae is selected from the group consisting of CICC1746 and BY4741.

16. An engineered strain synthesizing 24-epi-ergosterol, wherein the engineered strain is the engineered strain according to claim 1 comprising one or more of a sterol acylase ARE2, hydrolase YEH1 and hydrolase YEH2.

17. The engineered strain according to claim 15, wherein an original strain of the engineered strain is an ERG4 gene-knockout model strain CICC1746.

18. A method for producing 24-epi-ergosterol, comprising the following steps:
fermenting the engineered strain according to claim 11 in a fermentation medium.

19. The method according to claim 18, wherein the fermentation medium comprises a yeast extract-peptone-dextrose (YPD) culture medium.
